(19) United States
(12) Patent Application Publication
(10) Pub. No.: US 2022/0079615 A1
(43) Pub. Date: Mar. 17, 2022

(54) SURGICAL CUTTING APPARATUS FOR REMOVAL OF A TUMOUR FROM HUMAN TISSUE

(71) Applicant: I.M. Surgical AB, Växjö (SE)

(72) Inventor: Imad MOHAMMED, Växjö (SE)

(73) Assignee: I.M. Surgical AB, Växjö (SE)

(21) Appl. No.: 17/420,096

(22) PCT Filed: Jan. 14, 2020
(86) PCT No.: PCT/SE2020/050030

§ 371 Date: Jun. 30, 2021

(30) Foreign Application Priority Data
Feb. 8, 2019 (SE) 1950148-5

(51) Int. Cl. A61B 17/3205; A61B 18/14

(52) U.S. Cl. CPC A61B 17/32053; A61B 2017/008; A61B 2018/1412; A61B 18/14

(57) ABSTRACT

Surgical cutting apparatus (1) for removal of human tissue tumour. The apparatus (1) comprises a first knife (2) with a circular shaped side wall (5) comprising a cutting edge (6). The first knife (2) being arranged for cutting tissue in a first direction (7). The apparatus (1) comprises a shank (8), wherein the shank (8) has a width (w) of less than 15 millimetres. The first knife (2) has a height (h) of less than 15 millimetres. The shank (8) being a yoke comprising two legs (8a, 8b) interconnected at their respective second shank ends (10a, 10b). The apparatus (1) permitting removal of human tissue tumour by firstly bringing the first knife (2) under the skin through a narrow incision, and secondly cutting the tissue in the first direction (7) axially with the first knife (2), by an axially pressing and rotating movement of the first knife (2).

### TECHNICAL FIELD

The present invention relates generally to the domain of which a surgical method called lumpectomy belongs to. More specifically, the invention relates to a surgical cutting apparatus for removal of tumours from human tissue.

### BACKGROUND ART

Breast cancer is a common disease and is the most common cancer among women. Men can also get breast cancer even if it is unusual. The ratio is about 200-250 women to 1 man. The corner-stone of the treatment is surgery, of which an important part is so-called breast-preserving surgery (also known as lumpectomy or partial mastectomy). The other technique is to remove the hole breast, which is called mastectomy. The size and site of the tumour in the breast, and also the size of the breast determines which method may be used.

There are several names used for breast-preserving surgery such as biopsy, lumpectomy, partial mastectomy, re-excision, quadrantectomy, wedge or wide local excision. Lumpectomy is one method of partial mastectomy, and is a form of breast-conserving or breast preservation surgery, in which the breast tumour (the “lump”) and some of the normal tissue that surrounds it is removed. The amount of tissue removed can vary greatly. Quadrantectomy, for example, means that more breast will be removed than in the case of lumpectomy. All different kinds of methods for “breast-conserving” or “breast-preservation” surgery aims to completely remove the tumour and secure that also a part of healthy tissue around it is removed, to avoid so-called recurrence. The seeding effect is a term that means that cancer cells release and remain in the operative area, or its spread through bloodstream or to the lymph nodes. It is crucial for a successful surgery to get enough margin of healthy tissue around the tumour. Today the standard is so-called “no tumour on ink”, also known as “Negative Margins”, “Clean margins”, “Not involved margins” or “Clear margins”. The opposite is that cancer cells appear in the edges of the cut-out lumpectomy, and this is called “involved margin” or “positive margin”.

In lumpectomy, the surgeon normally operates with a kind of electric scalpel that uses heat to minimize bleeding (an electrocautery knife). It is also common to use curved incisions that follows the natural curve of the breast and allow for better healing. After lumpectomy, all the tissue removed from the breast is examined carefully to see if cancer cells are present in the margins as described above. If cancer cells are found in the margins of the breast tissue removed, the surgeon will perform additional surgery (called re-excision) to remove the remaining cancer cells. The reoperation can have many negative consequences. For instance it could cause a large tissue defect which is not easy to reconstruct, increased costs, psychological impact on patient and increased complications from anesthesia.

It is difficult to ensure the right margin because surgery is done more or less manually with a surgical knife, which means that the surgeon performs surgery according to his or her experience. One may follow the palpable tumour itself and try to excise it without touching it, or to follow a guide-wire for example in case of an nonpalpable tumour. Further, tumours are not completely symmetrical, i.e. have irregular forms, which involve greater difficulty in creating the right margins around the tumour. If the cut gets accidentally inclined it is easy to get to close to the tumour, and a clear margin may not be achieved. Other difficulties are for example that large breasts are difficult to operate because they often are “giggly”, which means that they move a lot and must be held tight during the surgery. This could be more demanding without a suitable surgical instrument.

The surgeon normally works by cutting multiple incisions around the tumour and releases it step by step. During the operation the surgeon normally holds the tumour with one hand and moves the tumour around to access around and below it. If the surgeon squeezes too much, one can theoretically cause a so-called seeding effect, as mentioned above, which means that cancer cells release and remain in the operative area, or is spread through bloodstream and lymph nodes.

The most difficult type of breast-conserving surgery is the so-called central lumpectomy, where the tumour lies just below the nipple. Since this type of operation is very difficult, most surgeons do not perform it. The technique demands great experience, and in reality there are few breast or plastic surgeons who have both skills, i.e. are able to perform both breast cancer surgery and plastic surgery, so called oncoplastic surgery. In central section surgery it is difficult to ensure clear margins and it is difficult to conduct the cosmetic operation. Instead, in most cases a mastectomy is performed directly. A successful plastic surgery for reshaping the breast requires that a sufficiently large volume of the breast remains. If re-excision is needed to secure clear margins, mastectomy will be the definitive treatment option. Nevertheless, the placement of incision on the breast is more challenging. The aim is to get the most acceptable esthetic results not only in preserving a fine breast contour but also to achieve the least possible visible scar.

### SUMMARY OF THE INVENTION

It is an object of the invention to address at least some of the problems and issues outlined above. It is possible to achieve these objects and others by using a surgical cutting apparatus as defined in the attached independent claims.

According to an aspect of the invention, a surgical cutting apparatus for removal of human tissue tumour is disclosed. The surgical cutting apparatus comprises a first knife, a shank and a handle. The first knife comprises a proximal end, a distal end and a circular shaped side wall arranged between the proximal end and the distal end. Further, the distal end comprises a cutting edge, arranged along at least a major portion of the distal end, and the first knife is arranged for cutting tissue in a first direction, which is an axial direction of the first knife. The cutting in the first direction is performed by an axially pressing and rotating movement of the first knife. The shank of the first knife comprises a first shank end and a second shank end, wherein the first shank end is connected to the proximal end of the first knife and has an axial extension away from the first knife. The shank has a width at the first shank end of less than 15 millimetres. The handle comprises a first handle end and a second handle end at a distance from the first handle end. The first handle end is connected to the second shank end. The apparatus permitting removal of human tissue tumour by firstly bringing the first knife under the skin through a narrow incision, facilitated by the narrow width of the shank, and secondly cutting the tissue in the first direction axially with the first knife, by an axially pressing and rotating movement of the first knife.

By such an arrangement it is possible to cut out a tube-shaped tissue with a very “clean” cut through a smaller incision and without a great risk of so-called seeding. By the rotating movement, preferably back and forth, together with a soft pressing force, and that the first knife has a cutting edge along at least a major portion of the distal end, the cut is very precise and well-controlled by the operating surgeon. Furthermore, the width of the shank at the first shank end which is less than 15 millimetres is thus relatively narrow. This means that it is possible to get under the skin without the need of creating a large incision in the skin, as the width of the shank is relatively narrow. The resulting excised tissue specimen is cylindrically shaped, excised through a much smaller incision in the skin due to the relatively narrow shank. Since the first shank end is connected to the proximal end of the first knife and that the shank protruding away from the first knife (i.e. in the opposite direction of the cutting direction of the first knife), and further is connected to the handle with its second end, a distance between the handle and the first knife is created by the shank. The narrow shank, causes an opening above the first knife and enables a great flexibility of controlling/directing the cut in the first direction, without affecting the precise cut negatively and without the need for large incisions, which is not possible in known prior art. By selecting the size of the diameter of the circular shaped side wall, depending on the size of the tumour, it is easy to get a “clear margin” around the tumour, and since the handling of the tumour is minimized by the unique design of the cutting apparatus, the seeding effect is minimized. With the invented cutting apparatus it is also easier to perform the most difficult type of breast-conserving surgery—the so-called central section, where the tumour lies just below the nipple.

According to an embodiment of the invention, the width of the shank at the first shank end is less than 10 millimetres and preferably between 1-7 millimetres.

An advantage with the solution, is that it is possible to get under the skin without the need of creating a large incision in the skin as the width of the shank at the first shank end is less than 10 millimetres and preferably between 1-7 millimetres and is thus even more relatively narrow.

According to an embodiment of the invention, the first knife has a height of less than 15 millimetres, preferably less than 10 millimetres and more preferably between 1-7 millimetres. The apparatus permitting removal of human tissue tumour by firstly bringing the first knife under the skin through a narrow incision, facilitated by the shorth height of the first knife, and secondly cutting the tissue in the first direction axially with the first knife, by an axially pressing and rotating movement of the first knife.

An advantage with the solution, is that it is possible to get under the skin without the need of creating a large incision in the skin, as the height of the first knife is relatively short together with the relatively narrow width of the shank. The resulting excised tissue specimen is cylindrically shaped, excised through a much smaller incision in the skin due to the relatively narrow shank and the relatively short height of the first knife. Furthermore, the short extension of the first knife (the circular blade) and the narrow shank, causes an opening above the first knife and enables a great flexibility of controlling/directing the cut in the first direction, without affecting the precise cut negatively and without the need for large incisions, which is not possible in known prior art.

According to an embodiment, the side wall accommodates a second knife for cutting in a second direction transverse to the first direction. By having a second knife incorporated in the side wall of the first knife or arranged in close connection to the side wall it is possible to perform the first cut in the first direction, and when a specific depth is achieved, that is below the tumour, perform a transverse cut relative to the first direction. For example, the first cut is performed in vertical direction, and at the correct depth in the tissue, a horizontal cut with the second knife is performed. As a result the specimen is detached from the rest of the tissue and the surgeon is then able to remove it from the body using the second knife only. Since the second knife is arranged inside or very close to the side wall, it doesn't interfere the first cut. By such an arrangement it is possible to cut out a complete tumour with a clear margin tissue around it and below it without handling the cut-out by hand and with a numerous of cuts around the tumour for removing it as in prior art. In prior art, the surgeon operates by cutting multiple incisions and step by step loosening the tumour all around by holding the tumour with one hand while simultaneously cutting around with a scalpel hold by the other hand. If the surgeon squeezes too much, one can cause the so-called seeding effect, where cancer cells release and remain in the operative area, or spread through bloodstream and lymph node. The inventive cutting apparatus minimizes the risk of seeding and the sharp and exact cuts makes it easier to examine the tissue, perform plastic surgery (if necessary) and also means a better healing of the wound, compared to prior art solutions.

According to another embodiment, the side wall comprises an opening arranged for the second knife, inside the side wall, to let the second knife be out of the way during the cut in the first direction by the first knife. The second knife is rotatable about a first end by that the first end is rotatably arranged in close connection to the side wall or inside the opening of the side wall. A second end of the second knife is arranged to rotate radially inward relative to the first knife. Since the first knife is designed as a short circular shaped knife, and the second knife is rotatable around the first end, the second end, which preferably is a free end, rotates inwards towards the centre of the first knife, and preferably passes the centre point of the first circular shaped knife. In this position, when the second knife has made the transverse cut relative the first cut, the first knife is rotated and by that is also the second knife rotated because of the attachment to the first knife, and any un-cut tissue will be cut in the transverse direction. It is now possible to lift he complete cut-out tissue, including the tumour, out from the breast. As the second knife is in its end position below the tumour, the second knife serves as a support under the tissue during the removal of the tissue from the breast.

According to another embodiment, the first end of the second knife is fixedly attached to an actuating shaft, and the actuating shaft is rotatably attached to the shank and extending axially along the first knife. Preferably, the side wall comprises a recess, in which a portion of the actuating shaft extends, and which recess preferably ends in connection to the opening in the side wall. By that, the actuating shaft is arranged for a minimum or no interference with the cut in the first direction, and is connected to the second knife, in the opening or in the end of the recess close to or inside the side wall. The actuating shaft is used for initiating and/or performing the cutting in the second direction by means of the second knife. Typically, the first knife is first of all positioned on the correct spot of the breast, above the tumour and is then pressed and rotated by the surgeon for performing the cut in the tissue in the first direction. When reached the correct depth, the second knife is activated by that the surgeon handles/actuates the rotatable actuating shaft, connected to second knife, and thus the cut in the second direction, transverse the first direction, is performed. In this way, the surgeon doesn't need to hold the tissues of the respective sides of the first cut apart and perform the second cut/cuts by a scalpel or the like. All surgery is performed by handling of the knives outside the cuts. An easy-handled and precise tool for completely removing of a human tissue tumour is thereby disclosed, which is not known in prior art.

According to an embodiment, the second knife is biased in its position in the opening of the side wall. By that the actuating shaft is attached to a first spring end of a spring, and further that the spring is attached with a second spring end to the handle or the shank, a spring force may be loaded to the actuating shaft. Thus, a rotation of the actuating shaft in one rotational direction, for example a clockwise rotational direction, is tensioning the spring. If the spring force is released, a rotation of the actuating shaft in a release rotational direction is achieved, which is opposite to the first “tensioning” rotational direction, in the example the counter-clockwise rotational direction. The releasing of the spring-force leads to the cutting of tissue in the second direction by means of the second knife. By such an arrangement, the cutting apparatus is very easy to manage, and the second cut is very distinct and precise, since the surgeon may hold the complete cutting apparatus very still, and anyway perform the second cut in a precise manner. This is performed without the need of pushing the tissue around for performing the second cut with a scalpel or the like, which in prior art solutions increases the risks of seeding and makes the cut rougher.

According to an embodiment the shape of the second knife substantially follows the shape of the cutting edge of the first knife, that is, the second knife has a curved shape. By this design, the second knife is easy to incorporate in the side wall without interfering the cut in the first direction, which is performed by the first knife.

According to yet another embodiment of the invention a tension-release device is arranged to tension as well as release the biasing force, i.e. the spring force, to initiate the cutting action of the second knife in the transverse direction relative to the first cutting direction. An easy-handled triggering of the second cut is preferred since the handling of the cutting apparatus must be easy, to control the cuts more precise. Thus, the second cut may be performed by using one hand only, which is a far better solution than known prior art solutions. And yet, the result is a more precise cut with all benefits as explained above.

According to an embodiment the shaft is a yoke comprising two legs interconnected at their respective second shank ends through a handle bracket, and the handle is in turn attached to the handle bracket. Preferably, the two legs are thin to not affect the cut and not to make a greater wound than necessary. The form of the shank is more or less like a stirrup, with the circular first knife connected to the handle by the two legs of the shank, which forms an open section between the first knife and the handle. This is as mentioned above very positive to make as small interference as possible on the skin-cut and the tissue-cut.

According to an embodiment the second knife is connected to an electrical source, to form a so-called electrocautery knife of the second knife. This may also be applicable to the first knife. Thus, a kind of electric cutting apparatus or electric knife, that uses heat to minimize bleeding, is disclosed.

According to an embodiment of the invention, the handle is formed as a shaft with pencil grip, and whose centre axis substantially coincides with the first knife centre axis. It is proven that a so-called pencil grip of the tool makes the handling of the cutting apparatus very precise, and that it is easy to direct the cutting in the right directions. It is also easy to get an overview of the surgery when the cutting tool is aligned around a centre axis.

According to yet another embodiment, the handle comprises grip enhancements, arranged for a better control of the cutting apparatus and a less slippery grip. The grip enhancements may be of any kind for example different patterns of various depth/height and dimensions.

According to an embodiment of the cutting apparatus, the second handle end of the handle comprises a knob, also for increasing the manoeuvring possibilities when using the surgical cutting apparatus. By the knob, it is easier to push the cutting apparatus in the first axial direction, because of the knob gives a support for the palm, but the knob is also beneficial during the combined pushing and rotation of the first knife during the cut in the first direction. The knob may have various design, for example a “mushroom-like” design with a wider diameter than the handle. Other designs are also possible.

According to an embodiment of the invention, the shank comprises a grading scale. For example, one or both legs comprise the scale, which purpose is to show the actual depth of the cut in the first direction, performed by the first knife. During preparations of the surgery, the tumour is localized in terms of area location and depth. When the cutting edge of the first knife has reached the correct depth for making a “clear margin” also in terms of depth, by the cut in the transverse direction, the surgeon actuates the second knife for the cut in the transverse direction. The scale is a very important tool for controlling the cut with the second knife, and the scale may be in various designs and gradings.

According to an embodiment, the handle is hollow. By a hollow handle, the total weight of the surgical cutting apparatus may be kept low, but it is also possible to include electrical wiring inside the handle, for example for activation of the second knife or for the having an electrical connection to the first and/or second knife.

Further possible features and benefits of this solution will become apparent from the detailed description below.

Surgical cutting apparatus according to claim 8, wherein a width (w) of each of the two legs (8a, 8b) at their respective first shank end (9a, 9b) is less than 15 millimetres.

Surgical cutting apparatus according to claim 8, wherein the width (w) of each of the two legs (8a, 8b) at their respective first shank end (9a, 9b) is less than 10 millimetres and preferably between 1-7 millimetres.

The second shank end (10) also has a width, wherein the width of the second shank end (10) is equal or greater than the width (w) of the first shank end (9).

As the circular shaped side wall preferably has a short extension between the distal end and the proximal end, it forms a relatively short tube-formed blade.

Method for use of a surgical cutting apparatus (1) according to any of the preceding claims for removal of human tissue tumour, the method comprising: axially pressing and rotating a first knife (2) for cutting tissue in a first direction (7) axially with the first knife (2), to a depth where at least a cutting edge (6) is below the tumour, actuate a second knife (13) for cutting tissue in a second direction (14) transverse to the first direction (7), by initiating a rotating movement of the second knife (13) about a first end (15), whereby a second end (16) of the second knife (13) rotates radially inward relative to the first knife (2) to an end position, rotate the first knife (2) for completely cutting eventually un-cut tissue in the second direction (14) when the second knife (13) is in its end position, lift the cut-out tissue in a substantially opposite direction relative to the first direction (7), for removal of the tissue comprising the tumour.

According to an aspect of the invention, a method for use of a surgical cutting apparatus according to any of the preceding claims for removal of human tissue tumour is disclosed. The method comprising the steps of first axially pressing and rotating a first knife for cutting tissue in a first direction, axially with the first knife and to a depth where at least a cutting edge of the first knife is below the tumour. The next step is to actuate a second knife for cutting tissue in a second direction transverse to the first direction. This is done by initiating a rotating movement of the second knife about a first end, whereby a second end of the second knife rotates radially inward relative to the first knife, to an end position of the second knife, which position is inside the circular shaped first knife. In this position of the first knife and second knife, the first knife is rotated for completely cutting eventually un-cut tissue in the second direction. This by that the second knife also rotates along with the first knife, since it is attached to the first knife with its first end. Now, the complete tissue including the tumour and clear margins is lifted in a substantially opposite direction relative to the first direction for removal of the cut-out piece of tissue comprising the tumour. The piece of tissue is lifted by means of the second knife, since it is in its end position, which is under the tissue inside the second knife. By the method, a safe way of removing tumours through small well-positioned skin incisions is achieved, which method also means less risk of seeding effect and rough edges of the cuts.

The method described is preceded by the steps of cutting a narrow incision in the skin, and bringing the first and the second knife under the skin through the narrow incision, facilitated by the narrow width of the shank and the short height of the first knife.

### BRIEF DESCRIPTION OF DRAWINGS

The solution will now be described in more detail by means of exemplary embodiments and with reference to the accompanying drawings, in which:

FIG. 1 is a perspective view of a cutting apparatus according to the invention. The cutting apparatus comprising a first knife, a shank and a handle, and is arranged for cutting in a first axial direction.

FIG. 2 is a perspective view of an embodiment of the cutting apparatus of FIG. 1. The cutting apparatus according to this embodiment further comprising a second knife and means for using/activating the second knife, arranged for cutting in a second direction, which is a transverse direction relative to the first axial direction. In the figure, the second knife is in a biased pre-surgery position in a side wall of the first knife.

FIG. 3 is a perspective view of an embodiment of the cutting apparatus of FIG. 2. In the figure, the second knife is in a released position inside the first knife.

FIG. 4 is a perspective view of an embodiment of the cutting apparatus of FIG. 2, wherein the second knife is connected to an electrical source.

### DETAILED DESCRIPTION

Briefly described, a surgical cutting apparatus is provided, that gives an efficient and well-functioning cutting apparatus, which is easy to handle and also provides a secure and precise removal of a tumour from human tissue through small well-positioned skin incisions. Further, it is easy to get a “clear margin” around the tumour, and since the handling of the tumour is minimized by the unique design of the cutting apparatus, the seeding effect is minimized.

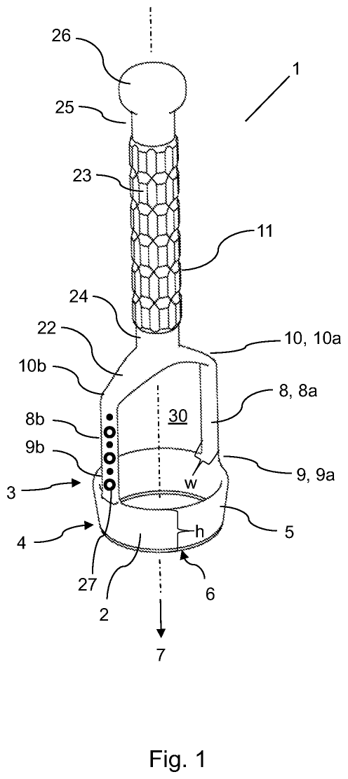

FIG. 1 shows a perspective view of a surgical cutting apparatus 1, for removal of human tissue tumour, which cutting apparatus comprises a first knife 2, a shank 8 and a handle 11, and which is arranged for cutting tissue in a first direction 7. The first knife 2, is designed like a circular, tube-shaped blade, with a proximal end 3, a distal end 4 and a circular shaped side wall 5 arranged between the proximal end 3 and the distal end 4. The distal end 4 comprises a cutting edge 6 along at least a major portion of the distal end 4. In the preferred embodiment, the cutting edge is a sharp edge, running continuously around the entire circumference of the distal end 4. The side wall 5 has preferably a short extension, why the distance between the proximal end 3 and distal end 4 is rather short. This design means that it is possible to get under the skin without the need of creating a large incision in the skin, and it is also easier to adjust the cutting direction during the first cut.

The proximal end 3 of the first knife 2 is connected to a shank 8. The shank 8 comprises a first shank end 9 connected to the first knife 2 and a second shank end 10 connected to the handle 11. In the preferred embodiment shown in FIG. 1, the shank 8 is designed as a yoke which comprises two legs 8a, 8b. The two legs 8a, 8b are preferably positioned opposite each other and are connected to the proximal end 3 of the first knife 2 with respective first shank ends 9a, 9b. Further, the two legs 8a, 8b extends from the first knife 2, and a distance from the first knife 2, they are interconnected at respective second shank ends 10a, 10b through a handle bracket 22. The distance of the shank 8/the legs 8a, 8b is crucial for creating an open area 30 above the first knife 2, only occupied by the two legs 8a, 8b, which preferably are thin, but strong enough for the surgery operation. The open area 30 together with the short extension of the side wall 5, enables good control of the direction of the cut with the first knife 2 in the first direction 7 and minimizes the need of a large incision. At least one of the legs 8a, 8b comprises a grading scale 27 which is crucial for determining the depth of the cut in the first direction 7. The scale and the grading may vary within the scope of invention.

The handle 11 comprises a first handle end 24 and a second handle end 26 at a distance from the first handle end 24, and the first handle end 24 is connected to the handle bracket 22. The handle 11 is designed as shaft with “pencil grip”, with a centre axis which substantially coincides with the centre axis of the first knife 2. Further, the handle 11 comprises grip enhancements 23 for a better control of the cutting apparatus 1 and a less slippery grip of the same. The grip enhancements 23 may be of any kind for example different patterns of various depth/height and dimensions. For even better control of the surgical cutting apparatus 1, the second handle end 25 comprises a knob 26. The knob 26 may for example have a design as in the preferred embodiment presented in FIG. 1, namely as a rounded knob 26 with larger diameter than the handle 11. The knob 26 forms a support for the palm of the surgeon, during the soft pressing of the cutting apparatus 1 in the first direction 7, as may also be used for a better control of the rotary movement of the cutting apparatus 1. Another possible feature of the handle 11, which is not visible in the figure, is that the handle preferably is hollow. Thus, the total weight of the cutting apparatus 1 gets lower and the cavity enables wiring and the like for use in connection of electrically coupled knifes etc.

The first knife 2 is thereby arranged for cutting tissue in the first direction 7, which is an axial direction of the surgical cutting apparatus 1. The cutting of tissue in the first direction 7 is performed by an axial pressing of the surgical cutting apparatus 1 in the first cutting direction 7 and simultaneously rotating the first knife 2 in one direction or back and forth.

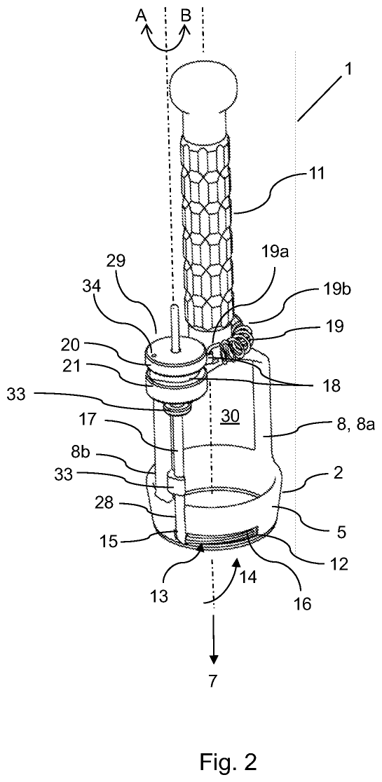

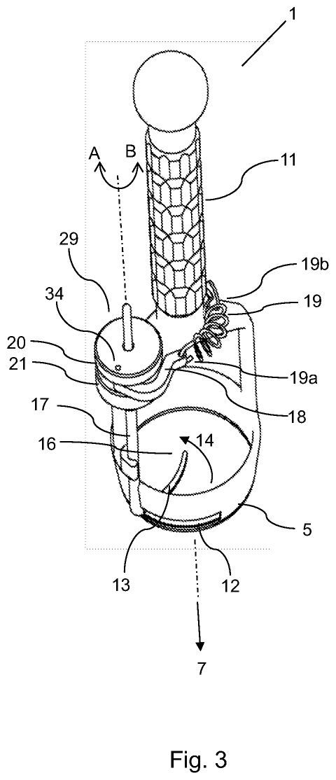

FIGS. 2 and 3 shows a preferred embodiment of the surgical cutting apparatus 1 of FIG. 1, which comprises a second knife 13, arranged for cutting tissue in a second direction 14, transverse to the cut in the first direction 7. In this embodiment, the side wall 5 of the first knife 2 comprises an opening 12, arranged to accommodate the second knife 13, which by that extends along the side wall 5. The second knife 13 is formed like the blade of a scythe and follows the form of the circular side wall 5 to be able to substantially fit in the opening 12. The second knife 13 is rotatable about a first end 15 by that the first end 15 is fixedly attached to an actuating shaft 17, which is rotatably attached to one of the legs 8a, 8b of the shank 8, by means of bearings 33. The angle between the actuating shaft 17 and the second knife 13 is preferably perpendicular or near perpendicular. A second end 16, which is a free end of the second knife 13 is arranged to rotate radially inward relative to the first knife 2, when actuated. The actuating shaft 17 extends axially along the leg 8a, 8b and thereby axially relative the first knife 2. Further, it is preferred that the side wall 5 comprises a groove 28, arranged to accommodate at least a part of the cross-section of the actuating shaft 17, such as the actuating shaft 17 do not interfere too much with cut in the first direction 7. The groove 28 ends in the opening 12, and the actuating shaft 17 and the second knife 13 is connected in the opening 12 near the groove 28.

To perform the cut in the second direction 14 in a manageable manner, the second knife 13 is biased in its position in the opening 12. The biasing of the second knife 13 is made by means of a spring 19, wherein a first spring end 19a of the spring 19 is attached directly or indirectly to the actuating shaft 17. In the preferred embodiment, the first spring end 19a is attached to a tension-release device 29, which comprises a first and a second wheel 20, 21 fixedly attached to the actuating shaft 17. The tension-release device 29 further comprises a hook 18, which is arranged between the wheels 20, 21. One end of the hook 18 is pivotally attached to a short axis 34, which extends between the first and the second wheel 20, 21 near the periphery of the wheels 20, 21. The first spring end 19a is attached to the other end of the hook 18. Further, the spring 19 is attached with a second spring end 19b to the handle 11 or the shank 8, for example to a bracket or the like. To pre-load the second knife 13 with the spring-force, a rotation of the first wheel 20, and thereby the actuating shaft 17, in a first rotational direction A is performed, which causes a tensioning of the spring 19 and thus, the second knife 13 is biased in its position in the side wall 5. In FIG. 2, a rotation of the first wheel 20 in a clockwise rotational direction, causes a rotation of the actuating shaft 17 and thereby also the second knife 13, which will move the second knife 13 from its end position inside the first knife 2 to its position in the side wall 5. At the same time the second knife 13 gets the biased state, since the tensioning of the spring 19 simultaneously occurs when rotating the first wheel 20/the actuating shaft 17 in the first rotational direction A. The rotating of the first wheel 20 in the first rotational direction A continues until the hook 18 passes a locking point, whereby the tension-release device 29 is locked and the second knife 13 is thereby spring-loaded and locked in its position in the side wall 5. Thus, the tension-release device 29 is arranged both for tensioning the spring 19 and for locking the actuating shaft 17/the second knife 13 in the biased position, as well as arranged for releasing the spring 13, to perform the cut in the second direction 14, by means of the second knife 13. The release of the biasing force is initiated by that the surgeon turns the first wheel 20 of the tension-release device 29 in a second rotational direction B, which is an opposite rotational direction relative to the first rotational direction A, whereby the spring-force is released when the locking position is passed, and a springed rotation of the actuating shaft 17 in the second rotational direction B occurs. Thus, the second knife 13 rotates radially inward relative to the first knife 2, and referring to FIG. 2 this is a counter-clockwise direction and passes the centre of the tumour. After the second knife 13 has made the cut in the second direction 14, the first knife 13 is rotated in the first direction 7 (clockwise) to free the tumour completely. After this the tumour may be lifted and the second knife 13 serves as a support during the lifting motion.

The cutting edge 6 of the first knife 2 as well as the second knife 13, or a cutting edge of the second knife 13, is made of stainless steel of a special type which is common for surgical knives. All other parts of both the first and second knives 2, 13 may be of metal or plastic or the like. For example, the handle 11 with the knob 26, the shank 8 with the legs 8a, 8b and the handle bracket 22, the first knife 2 excluding the cutting edge 6, but including the side wall 5 and proximal end 3, and the actuating shaft 17 with the first and second brackets 18, 22 may be in plastic material. The spring 19 is preferably a standard spring 19 made of metal.

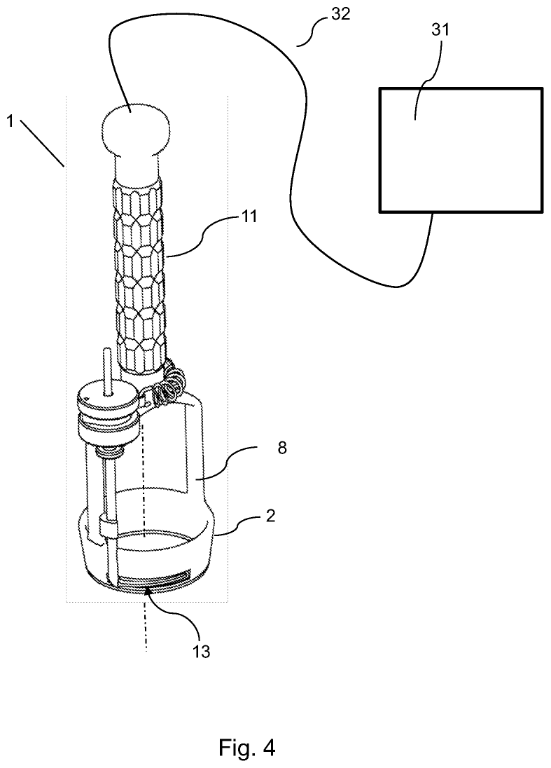

FIG. 4 shows a preferred embodiment of the surgical cutting apparatus 1 of FIGS. 2 and 3, wherein the second knife 13 is connected to an electrical source 31, to utilizing the technique of so-called electrocautery knife surgery, for the use of heat to minimize bleeding. The preferred embodiment also comprises electrical wiring by that a wire 32 is connected to the second knife 13 and the electrical source 31 and which wire 32 is arranged inside the hollow handle 11. If the second knife 13 is connected to an electrical source 31, some metal parts of the second knife 13 may have to be isolated with a heat resistant rubber-gasket or the like, in order to decrease the risk of unnecessarily burning human flesh.

Other possible embodiments (not visible in any figure) could be for example that the cutting edge 6 of the first knife 2 have a wave form, and that the cutting edge 6 is a plurality of shorter cutting edges arranged along the circumference of the distal end 4, and finally that the cutting edge 6 is detachable by a click-function or the like.

Although the description above contains a plurality of specificities, these should not be construed as limiting the scope of the concept described herein but as merely providing illustrations of some exemplifying embodiments of the described concept. It will be appreciated that the scope of the presently described concept fully encompasses other embodiments which may become obvious to those skilled in the art, and that the scope of the presently described concept is accordingly not to be limited. Reference to an element in the singular is not intended to mean “one and only one” unless explicitly so stated, but rather “one or more.” All structural and functional equivalents to the elements of the above-described embodiments that are known to those of ordinary skill in the art are expressly incorporated herein and are intended to be encompassed hereby.

1. Surgical cutting apparatus (1) for removal of human tissue tumour, comprising:
a first knife (2), comprising a proximal end (3), a distal end (4) and a circular shaped side wall (5) arranged between the proximal end (3) and the distal end (4), wherein the distal end (4) comprising a cutting edge (6) along at least a major portion of the distal end (4), wherein the first knife (2) being arranged for cutting tissue in a first direction (7) axially with the first knife (2), by an axially pressing and rotating movement of the first knife (2),
a shank (8), comprising a first shank end (9) and a second shank end (10), wherein the first shank end (9) being connected to the proximal end (3) and having an axial extension away from the first knife (2),
wherein the shank (8) being a yoke comprising two legs (8a, 8b) interconnected at their respective second shank ends (10a, 10b),
wherein the shank (8) has a width (w) at the first shank end (9) of less than 15 millimetres,
wherein the first knife (2) has a height (h) of less than 15 millimetres,
a handle (11), comprising a first handle end (24) and a second handle end (25) at a distance from the first handle end (24), wherein the first handle end (24) being connected to the second shank end (10),
the apparatus (1) permitting removal of human tissue tumour by firstly bringing the first knife (2) under the skin through a narrow incision, facilitated by the narrow width (w) of the shank (8) and the short height (h) of the knife (2), and secondly cutting the tissue in the first direction (7) axially with the first knife (2), by an axially pressing and rotating movement of the first knife (2).

2. The surgical cutting apparatus according to claim 1, wherein the width (w) of the shank (8) at the first shank end (9) is less than 10 millimetres and preferably between 1-7 millimetres.

3. The surgical cutting apparatus according to claim 1, wherein the first knife (2) has a height (h) of preferably less than 10 millimetres and more preferably between 1-7 millimetres, the apparatus (1) permitting removal of human tissue tumour by firstly bringing the first knife (2) under the skin through a narrow incision, facilitated by the shorth height (h) of the first knife (2), and secondly cutting the tissue in the first direction (7) axially with the first knife (2), by an axially pressing and rotating movement of the first knife (2).

4. The surgical cutting apparatus according to claim 1, wherein the side wall (5) comprises a second knife (13) for cutting in a second direction (14) transverse to the first direction (7).

5. The surgical cutting apparatus according to claim 4, wherein the side wall (5) comprises an opening (12) arranged to accommodate the second knife (13), and the second knife (13) is rotatable about a first end (15) by that the first end (15) of the second knife (13) is rotatably arranged in close connection to the side wall (5), and a second end (16) of the second knife (13) is arranged to rotate radially inward relative to the first knife (2).

6. The surgical cutting apparatus according to claim 5, wherein the first end (15) of the second knife (13) being fixedly attached to an actuating shaft (17), wherein the actuating shaft (17) being rotatably attached to the shank (8) and extending axially along the first knife (2).

7. The surgical cutting apparatus according to claim 6, wherein the second knife (13) is biased in its position in the opening (12) of the side wall (5), wherein the actuating shaft (17) is connected to a first spring end (19a) of a spring (19), wherein the spring (19) further is attached to the handle (11) or to the shank (8) by means of a second spring end (19b) of the spring (19), whereby a rotation of the actuating shaft (17) in a first rotational direction (A) tensioning the spring (19), and a releasing of the spring (19) causes a rotation of the actuating shaft (17) in a second rotational direction (B), which is opposite to the first rotational direction (A), and by that the rotation of the second knife (13) for cutting in the second direction (14).

8. The surgical cutting apparatus according to claim 4, wherein the shape of the second knife (13) substantially following the shape of the cutting edge (6) of the first knife (2), that is, having a curved shape.

9. The surgical cutting apparatus according to claim 4, wherein a tension-release device (29) is arranged to tension and to release the biasing force I spring force, for initiating the cutting action of the second knife (13).

10. The surgical cutting apparatus according to claim 1, wherein the two legs (8a, 8b) are interconnected through a handle bracket (22), and the handle (11) being attached to the handle bracket (22) with its first handle end (24).

11. The surgical cutting apparatus according to claim 4, wherein the second knife (13) being connected to an electrical source (31) by a wire (32).

12. The surgical cutting apparatus according to claim 1, wherein the handle (11) being formed as a shaft with pencil grip, and whose centre axis substantially coincides with the first knife (2) centre axis.

13. The surgical cutting apparatus according to claim 1, wherein the handle (11) comprising grip enhancements (23).

14. The surgical cutting apparatus according to claim 1, wherein the second handle end (25) comprising a knob (26).

15. The surgical cutting apparatus according to claim 1, wherein the shank (8) comprising a grading scale (27).

16. The surgical cutting apparatus according to claim 1, wherein the handle (11) being hollow.
